(19) United States
(12) Patent Application Publication
(10) Pub. No.: US 2026/0265286 A1
(43) Pub. Date: Sep. 10, 2026

(54) PROCESS FOR PREPARING MONOSACCHARIDE SOLUTION FROM STRAW-TYPE LINGOCELLULOSE BY GRADIENT ACID TREATMENT

(71) Applicant: NANJING TECH UNIVERSITY, Nanjing (CN)

(72) Inventors: Chenjie ZHU, Nanjing (CN); Yulian CAO, Nanjing (CN); Hanjie YING, Nanjing (CN); Yanjun CHEN, Nanjing (CN); Jie DAI, Nanjing (CN); Mingyu ZHU, Nanjing (CN); Xiaoyi ZHAI, Nanjing (CN)

(73) Assignee: NANJING TECH UNIVERSITY, Nanjing (CN)

(21) Appl. No.: 19/663,816

(22) Filed: Apr. 30, 2026

Related U.S. Application Data
(63) Continuation of application No. PCT/CN2024/082102, filed on Mar. 18, 2024.

(30) Foreign Application Priority Data
Nov. 7, 2023 (CN) 202311470429.2

(51) Int. Cl. C07H 1/08; C07H 3/02

(52) U.S. Cl. CPC C07H 1/08; C07H 3/02

(57) ABSTRACT

Disclosed is a process for preparing a monosaccharide solution from a straw-type lignocellulose by gradient acid treatment, including the following steps: S1: subjecting a lignocellulosic biomass to a first acidolysis reaction catalyzed by an acid solution with a concentration of more than 50 wt % to obtain a first acidolysis liquid; S2: evenly mixing the first acidolysis liquid with an organic solvent, and performing solid-liquid separation to obtain a glycan-containing residue; and S3: adding water and/or an acid solution to the glycan-containing residue to obtain a mixture with an acid concentration of less than 45 wt %, and performing a second acidolysis reaction to obtain a monosaccharide-containing reaction liquid. In the invention, the pretreatment is carried out at room temperature and atmospheric pressure without additional energy input, and all reagents used in the pretreatment process are recyclable, which greatly reduces the production cost.

### CROSS-REFERENCE TO RELATED APPLICATIONS

This application is the U.S. continuation application of International Application No. PCT/CN2024/082102 filed on 18 Mar. 2024 which designated the U.S. and claims priority to Chinese Application No. CN202311470429.2 filed on 7 Nov. 2023, the entire contents of each of which are hereby incorporated by reference.

### TECHNICAL FIELD

The present invention belongs to the fields of biochemical engineering and bioenergy, and relates to a process for preparing a monosaccharide solution from a straw-type lignocellulose by gradient acid treatment.

### BACKGROUND

Due to high emissions of greenhouse gases, bioenergy derived from a lignocellulosic biomass is regarded as a suitable and potential alternative to fossil fuels. Agricultural, forestry and industrial sectors are the main contributors to the availability of the lignocellulosic biomass, and the most promising raw materials are crop residues and forest residues. The lignocellulosic biomass becomes a potential source for the production of biofuels and biochemical products due to the abundance and low cost.

A lignocellulosic biomass material is composed of three polymer layers, including cellulose, hemicellulose and lignin, and because of being a complex aromatic polymer, the lignin hinders the process efficiency during the production of biofuels and biochemical products. The lignin component can be effectively removed by a pretreatment technology to reduce a negative impact. However, traditional pretreatment inevitably leads to problems such as a carbohydrate loss, and the residues obtained after pretreatment still requires enzyme introduction to obtain a monosaccharide component, resulting in high costs for biorefinery. In traditional pretreatment processes, physical pretreatment alone has little effect and high-load operating costs, while physical and chemical pretreatment requires a high temperature, a dedicated reactor and an expensive instrument, and biological pretreatment has a long cycle, during which a part of carbohydrates is consumed due to the utilization for microbial growth.

To overcome key problems encountered in the preparation of monosaccharide from the lignocellulosic biomass, many researchers have proposed corresponding solutions. For example, the Chinese Invention CN115341004A discloses a method for preparing a monosaccharide from a biomass raw material. However, in this method, an enzyme preparation requires to be added in two stages for preparing the monosaccharide, and meanwhile, the lignocellulosic biomass is treated by at least one of steam explosion, cooking and extrusion before the use of the enzyme preparation, resulting in a high overall process cost, a long cycle and complicated operations. The Chinese Invention CN101855368A discloses a method for preparing a sugar product. However, in this method, it is necessary to maintain a high temperature and a high pressure during acid treatment, which brings certain challenges to the pretreatment process. The Chinese Invention CN114410850A discloses a comprehensive method for treating a lignocellulosic material. However, in this method, high energy consumption is required, and meanwhile, the recycle and reuse of acid used in the pretreatment process are ignored.

Therefore, the development of an economical, efficient and green method to obtain a monosaccharide solution remains a key challenge for a lignocellulose conversion technology.

### SUMMARY

The technical problem to be solved by the present invention is to provide a process for preparing a monosaccharide solution from a straw-type lignocellulose by gradient acid treatment, aiming at the defects in the prior art, so as to solve the problem of sugar degradation into by-products that affects a saccharification yield and generates a microbial fermentation inhibitor during conventional non-grain bio-based sugar production, and the problem of energy consumption under harsh high-temperature and high-pressure conditions.

In order to solve the above technical problems, the following technical solution is used in the present invention.

The present invention discloses a process for preparing a monosaccharide solution from a straw-type lignocellulose by gradient acid treatment, including the following steps:
S1: subjecting the lignocellulosic biomass to a first acidolysis reaction catalyzed by an acid solution with a concentration of more than 50 wt % to obtain a first acidolysis liquid;
S2: evenly mixing the first acidolysis liquid with an organic solvent, and performing solid-liquid separation to obtain a glycan-containing residue;
S3: adding water and/or an acid solution to the glycan-containing residue to obtain a mixture with an acid concentration of less than 45 wt %, performing a second acidolysis reaction to obtain a monosaccharide-containing reaction liquid, and performing solid-liquid separation to obtain a monosaccharide-containing solution and a lignin precipitate; and treating the monosaccharide-containing solution by a simulated moving bed to obtain a monosaccharide liquid and an acid solution; and
S4: adding water to an acid-containing liquid phase obtained after the solid-liquid separation in the step S2 for back-extraction, and phase separation to obtain an acid solution and an organic phase; purifying the organic phase obtained and then recycling the organic phase back to the step S2 as the organic solvent; concentrating the acid solution obtained (including the acid solution in the step S3) to obtain an acid solution with a concentration of more than 50 wt % and condensed water or a dilute acid solution; recycling the acid solution with the concentration of more than 50 wt % obtained back to the step S1 as the acid solution; and recycling the condensed water or the dilute acid solution obtained back to the step S3.

In the step S1, the lignocellulosic biomass comprises a straw-type lignocellulosic biomass; and preferably, the straw-type lignocellulosic biomass is any one or a combination of several of wheat straw, corn stover, rice straw, sorghum straw, bagasse, corn cob, switchgrass, reed and rapeseed, and preferably the wheat straw.

In the step S1, the aqueous acid solution is an aqueous solution of an inorganic acid; preferably, the inorganic acid is any one or a combination of several of sulfuric acid, hydrochloric acid and phosphoric acid; and preferably, an acid concentration in the acid solution is 55 wt %-95 wt %, preferably 60 wt %-90 wt %, and preferably 80 wt %.

In the step S1, a mass ratio of the lignocellulosic biomass to an acid in the acid solution is 1:0.5-2.5, preferably 1: 1-2, and preferably 1:1.5.

In the step S1, a reaction temperature of the first acidolysis reaction is 20-30° C., and preferably room temperature.

In the step S1, a pressure of the first acidolysis reaction is atmospheric pressure.

In the step S1, the first acidolysis reaction is terminated when the system has good fluidity, and taking 10 kg of straw as an example, the reaction requires to last for 1 hour.

In the step S2, the organic solvent is any one or a combination of several of alcoholic organic solvent, ketone organic solvent and tertiary amine organic solvent.

In the step S2, a mass ratio of the first acidolysis liquid to the organic solvent is 1: 1-5, preferably 1: 2-4, preferably 1:2.5-3.5, and preferably 1:3.

In the step S2, the purpose of mixing the first acidolysis liquid with the organic solvent is to extract and recycle the acid from the first acidolysis liquid, and a number of extractions is not limited, and is adjusted according to actual conditions.

In the step S3, the acid concentration of the mixture is 5 wt %-35 wt %, preferably 10 wt %-30 wt %, and preferably 20 wt %.

In the step S3, a temperature of the second acidolysis reaction is 60-120° C., preferably 70-110° C., preferably 80-100° C., and preferably 90° C.

In the step S3, a pressure of the second acidolysis reaction is atmospheric pressure.

In the step S3, the second acidolysis reaction lasts for 0.3-2.5 hours, preferably 0.4-2 hours, preferably 0.5-1.5 hours, and preferably 1 hour.

The glycan in the present invention mainly includes glucan and xylan.

### Beneficial Effects

In the present invention, the straw-type lignocellulosic biomass is first pretreated (i.e., the first acidolysis reaction) at room temperature and atmospheric pressure without additional energy input, and all reagents used in the pretreatment process are recyclable, which greatly reduces the production cost.

In the present invention, the straw-type lignocellulosic biomass is first subjected to preliminary pretreatment (which is primary acidolysis) at room temperature and atmospheric pressure, and then subjected to secondary acidolysis, and the resulting monosaccharide conversion rate is significantly higher than that obtained by conventional thermal treatment, thereby offering a new approach for the development of a non-grain bio-based sugar platform.

In the present invention, a relatively low liquid-solid ratio is used, the monosaccharide can be directly obtained after the pretreatment, and finally, a monosaccharide solution with a high and adjustable concentration is obtained, thereby avoiding a large investment in enzyme costs.

### BRIEF DESCRIPTION OF THE DRAWINGS

The present invention is further described in detail hereinafter with reference to the drawings and specific embodiments, and the advantages of the above and/or other aspects of the present invention will be clearer.

FIG. 1 is a flow chart of pretreatment of a straw-type lignocellulosic biomass in an embodiment of the present invention.

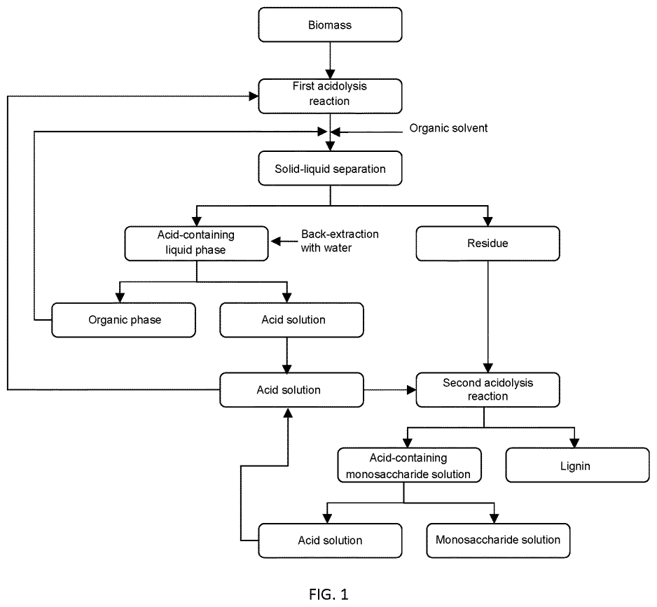

FIG. 2 is a two-dimensional aromatic NMR (nuclear magnetic resonance) spectrum of a lignin obtained in Embodiment 11 of the present invention.

FIG. 3 shows types and distribution of glycans obtained by primary acidolysis in Embodiment 11 of the present invention.

FIG. 4 shows a monosaccharide solution obtained in Embodiment 11 of the present invention.

### DETAILED DESCRIPTION

All experimental methods described in the following embodiments are conventional methods unless otherwise specified; and all reagents and materials are commercially available unless otherwise specified.

A straw-type lignocellulosic biomass raw material used in the following embodiments is wheat straw, and in the wheat straw, a cellulose content is 34.92 wt %, a hemicellulose content is 22.58 wt %, and a lignin content is 23.03 wt %.

Treatment conditions by a simulated moving bed in the following embodiments are as follows: strong-acid styrene-based hydrogen-form resin is used, a circulation flow rate is 75 mL/min, a feed flow rate is 25 mL/min, an eluent is water, and an elution flow rate is 75 mL/min.

Calculation methods for a glucose conversion rate and a xylose conversion rate in the embodiments of the present invention are as follows:

$$cellulose conversion rate \% = {C}_{G} * V / {M}_{G} * 100 \%$$

$$hemicellulose conversion rate \% = {C}_{X} * V / {M}_{X} * 100 \%$$

C_{G}: a concentration of glucose in the monosaccharide solution; C_{X}: a concentration of xylose in the monosaccharide solution; V: a volume of the monosaccharide solution; M_{G}: a mass of glucose in the straw used in the reaction; and M_{X}: a mass of xylose in the straw used in the reaction.

### Embodiment 1

A method for obtaining an acid-containing monosaccharide solution from a straw-type lignocellulosic biomass by acid treatment included the following steps.

In S1, wheat straw (40-100 mesh) was mixed with an aqueous sulfuric acid solution at a mass concentration of 60 wt %, and the wheat straw could be added in batches or in one portion, wherein a mass ratio of the wheat straw to the aqueous sulfuric acid solution was 1:1. The reaction liquid was subjected to first acidolysis reaction at room temperature and atmospheric pressure until the reaction liquid had good fluidity, so as to obtain a first acidolysis liquid.

In S2, the first acidolysis liquid obtained in the step S1 was fully mixed with 1-heptanol, wherein a mass ratio of the first acidolysis liquid to the 1-heptanol was 1:3. After uniform mixing, solid-liquid separation was performed to obtain an acid-containing liquid phase and a glycan-containing and lignin-containing residue.

In S3, water and/or an acid solution were added to the glycan-containing and lignin-containing residue obtained in the step S2 for mixing to obtain a secondary acidolysis mixture (with an acid concentration of 10 wt %), and then, secondary acidolysis reaction was performed at 80° C. and atmospheric pressure for 0.5 hour. After the acid reaction, solid-liquid separation was performed to obtain an acid-containing monosaccharide solution and a lignin precipitate. The acid-containing monosaccharide solution obtained was treated by a simulated moving bed to obtain a monosaccharide solution and an acid solution.

In S4, water was added to the acid-containing liquid phase obtained in the step S2 for back-extraction, and the mixture was allowed to stand for phase separation to obtain an acid solution and an organic phase. The organic phase obtained was purified and then recycled back to the step S2 as the organic solvent. The acid solution obtained (including the acid solution obtained in the step S3) was concentrated to obtain an acid solution with a concentration of more than 60 wt % and condensed water or a dilute acid solution; the dilute acid solution of more than 60 wt % obtained was recycled back to the step S1 as the acid solution; and the condensed water or the dilute acid solution obtained was recycled back to the step S3 as a secondary acidolysis solvent.

After two-stage acid hydrolysis, a glucose conversion rate of 71.36% and a xylose conversion rate of 63.14% could be achieved.

### Embodiment 2

A method for obtaining an acid-containing monosaccharide solution from a straw-type lignocellulosic biomass by acid treatment included the following steps.

In S1, wheat straw (40-100 mesh) was mixed with an aqueous sulfuric acid solution at a mass concentration of 60 wt %, and the wheat straw could be added in batches or in one portion, wherein a mass ratio of the wheat straw to the aqueous sulfuric acid solution was 1:1. The reaction liquid was subjected to first acidolysis reaction at room temperature and atmospheric pressure until the reaction liquid had good fluidity, so as to obtain a first acidolysis liquid.

In S2, the first acidolysis liquid obtained in the step S1 was fully mixed with methyl ethyl ketone, wherein a mass ratio of the first acidolysis liquid to the methyl ethyl ketone was 1:3. After uniform mixing, solid-liquid separation was performed to obtain an acid-containing liquid phase and a glycan-containing and lignin-containing residue.

In S3, water and/or an acid solution were added to the glycan-containing and lignin-containing residue obtained in the step S2 for mixing to obtain a secondary acidolysis mixture (with an acid concentration of 10 wt %), and then, secondary acidolysis reaction was performed at 80° C. and atmospheric pressure for 0.5 hour. After the acid reaction, solid-liquid separation was performed to obtain an acid-containing monosaccharide solution and a lignin precipitate. The acid-containing monosaccharide solution obtained was treated by a simulated moving bed to obtain a monosaccharide solution and an acid solution.

In S4, water was added to the acid-containing liquid phase obtained in the step S2 for back-extraction, and the mixture was allowed to stand for phase separation to obtain an acid solution and an organic phase. The organic phase obtained was purified and then recycled back to the step S2 as the organic solvent. The acid solution obtained (including the acid solution obtained in the step S3) was concentrated to obtain an acid solution with a concentration of more than 60 wt % and condensed water or a dilute acid solution; the acid solution with the concentration of more than 60 wt % obtained was recycled back to the step S1 as the acid solution; and the condensed water or the dilute acid solution obtained was recycled back to the step S3 as a secondary acidolysis solvent.

After two-stage acid hydrolysis, a glucose conversion rate of 66.89% and a xylose conversion rate of 52.66% could be achieved.

### Embodiment 3

A method for obtaining an acid-containing monosaccharide solution from a straw-type lignocellulosic biomass by acid treatment included the following steps.

In S1, wheat straw (40-100 mesh) was mixed with an aqueous sulfuric acid solution at a mass concentration of 60 wt %, and the wheat straw could be added in batches or in one portion, wherein a mass ratio of the wheat straw to the aqueous sulfuric acid solution was 1:1. The reaction liquid was subjected to first acidolysis reaction at room temperature and atmospheric pressure until the reaction liquid had good fluidity, so as to obtain a first acidolysis liquid.

In S2, the first acidolysis liquid obtained in the step S1 was fully mixed with tris(2-ethylhexyl)amine, wherein a mass ratio of the first acidolysis liquid to the tris(2-ethylhexyl)amine was 1:3. After uniform mixing, solid-liquid separation was performed to obtain an acid-containing liquid phase and a glycan-containing and lignin-containing residue.

In S3, water and/or an acid solution were added to the glycan-containing and lignin-containing residue obtained in the step S2 for mixing to obtain a secondary acidolysis mixture (with an acid concentration of 10 wt %), and then, secondary acidolysis reaction was performed at 80° C. and atmospheric pressure for 0.5 hour. After the acid reaction, solid-liquid separation was performed to obtain an acid-containing monosaccharide solution and a lignin precipitate. The acid-containing monosaccharide solution obtained was treated by a simulated moving bed to obtain a monosaccharide solution and an acid solution.

In S4, water was added to the acid-containing liquid phase obtained in the step S2 for back-extraction, and the mixture was allowed to stand for phase separation to obtain an acid solution and an organic phase. The organic phase obtained was purified and then recycled back to the step S2 as the organic solvent. The acid solution obtained (including the acid solution obtained in the step S3) was concentrated to obtain an acid solution with a concentration of more than 60 wt % and condensed water or a dilute acid solution; the acid solution with the concentration of more than 60 wt % obtained was recycled back to the step S1 as the acid solution; and the condensed water or the dilute acid solution obtained was recycled back to the step S3 as a secondary acidolysis solvent.

After two-stage acid hydrolysis, a glucose conversion rate of 77.24% and a xylose conversion rate of 68.62% could be achieved.

### Embodiment 4

A method for obtaining an acid-containing monosaccharide solution from a straw-type lignocellulosic biomass by acid treatment included the following steps.

In S1, wheat straw (40-100 mesh) was mixed with an aqueous sulfuric acid solution at a mass concentration of 90 wt %, and the wheat straw could be added in batches or in one portion, wherein a mass ratio of the wheat straw to the aqueous sulfuric acid solution was 1:2. The reaction liquid was subjected to first acidolysis reaction at room temperature and atmospheric pressure until the reaction liquid had good fluidity, so as to obtain a first acidolysis liquid.

In S2, the first acidolysis liquid obtained in the step S1 was fully mixed with 1-heptanol, wherein a mass ratio of the first acidolysis liquid to the 1-heptanol was 1:3. After uniform mixing, solid-liquid separation was performed to obtain an acid-containing liquid phase and a glycan-containing and lignin-containing residue.

In S3, water and/or an acid solution were added to the glycan-containing and lignin-containing residue obtained in the step S2 for mixing to obtain a secondary acidolysis mixture (with an acid concentration of 30 wt %), and then, secondary acidolysis reaction was performed at 100° C. and atmospheric pressure for 1.5 hour. After the acid reaction, solid-liquid separation was performed to obtain an acid-containing monosaccharide solution and a lignin precipitate. The acid-containing monosaccharide solution obtained was treated by a simulated moving bed to obtain a monosaccharide solution and an acid solution.

In S4, water was added to the acid-containing liquid phase obtained in the step S2 for back-extraction, and the mixture was allowed to stand for phase separation to obtain an acid solution and an organic phase. The organic phase obtained was purified and then recycled back to the step S2 as the organic solvent. The acid solution obtained (including the acid solution obtained in the step S3) was concentrated to obtain an acid solution with a concentration of more than 90 wt % and condensed water or a dilute acid solution; the acid solution with the concentration of more than 90 wt % obtained was recycled back to the step S1 as the acid solution; and the condensed water or the dilute acid solution obtained was recycled back to the step S3 as a secondary acidolysis solvent.

After two-stage acid hydrolysis, a glucose conversion rate of 68.56% and a xylose conversion rate of 54.38% could be achieved.

### Embodiment 5

A method for obtaining an acid-containing monosaccharide solution from a straw-type lignocellulosic biomass by acid treatment included the following steps.

In S1, wheat straw (40-100 mesh) was mixed with an aqueous sulfuric acid solution at a mass concentration of 90 wt %, and the wheat straw could be added in batches or in one portion, wherein a mass ratio of the wheat straw to the aqueous sulfuric acid solution was 1:2. The reaction liquid was subjected to first acidolysis reaction at room temperature and atmospheric pressure until the reaction liquid had good fluidity, so as to obtain a first acidolysis liquid.

In S2, the first acidolysis liquid obtained in the step S1 was fully mixed with methyl ethyl ketone, wherein a mass ratio of the first acidolysis liquid to the methyl ethyl ketone was 1:3. After uniform mixing, solid-liquid separation was performed to obtain an acid-containing liquid phase and a glycan-containing and lignin-containing residue.

In S3, water and/or an acid solution were added to the glycan-containing and lignin-containing residue obtained in the step S2 for mixing to obtain a secondary acidolysis mixture (with an acid concentration of 30 wt %), and then, secondary acidolysis reaction was performed at 100° C. and atmospheric pressure for 1.5 hour. After the acid reaction, solid-liquid separation was performed to obtain an acid-containing monosaccharide solution and a lignin precipitate. The acid-containing monosaccharide solution obtained was treated by a simulated moving bed to obtain a monosaccharide solution and an acid solution.

In S4, water was added to the acid-containing liquid phase obtained in the step S2 for back-extraction, and the mixture was allowed to stand for phase separation to obtain an acid solution and an organic phase. The organic phase obtained was purified and then recycled back to the step S2 as the organic solvent. The acid solution obtained (including the acid solution obtained in the step S3) was concentrated to obtain an acid solution with a concentration of more than 90 wt % and condensed water or a dilute acid solution; the acid solution with the concentration of more than 90 wt % obtained was recycled back to the step S1 as the acid solution; and the condensed water or the dilute acid solution obtained was recycled back to the step S3 as a secondary acidolysis solvent.

After two-stage acid hydrolysis, a glucose conversion rate of 62.37% and a xylose conversion rate of 50.14% could be achieved.

### Embodiment 6

A method for obtaining an acid-containing monosaccharide solution from a straw-type lignocellulosic biomass by acid treatment included the following steps.

In S1, wheat straw (40-100 mesh) was mixed with an aqueous sulfuric acid solution at a mass concentration of 90 wt %, and the wheat straw could be added in batches or in one portion, wherein a mass ratio of the wheat straw to the aqueous sulfuric acid solution was 1:2. The reaction liquid was subjected to first acidolysis reaction at room temperature and atmospheric pressure until the reaction liquid had good fluidity, so as to obtain a first acidolysis liquid.

In S2, the first acidolysis liquid obtained in the step S1 was fully mixed with tris(2-ethylhexyl)amine, wherein a mass ratio of the first acidolysis liquid to the tris(2-ethylhexyl)amine was 1:3. After uniform mixing, solid-liquid separation was performed to obtain an acid-containing liquid phase and a glycan-containing and lignin-containing residue.

In S3, water and/or an acid solution were added to the glycan-containing and lignin-containing residue obtained in the step S2 for mixing to obtain a secondary acidolysis mixture (with an acid concentration of 30 wt %), and then, secondary acidolysis reaction was performed at 100° C. and atmospheric pressure for 1.5 hour. After the acid reaction, solid-liquid separation was performed to obtain an acid-containing monosaccharide solution and a lignin precipitate. The acid-containing monosaccharide solution obtained was treated by a simulated moving bed to obtain a monosaccharide solution and an acid solution.

In S4, water was added to the acid-containing liquid phase obtained in the step S2 for back-extraction, and the mixture was allowed to stand for phase separation to obtain an acid solution and an organic phase. The organic phase obtained was purified and then recycled back to the step S2 as the organic solvent. The acid solution obtained (including the acid solution obtained in the step S3) was concentrated to obtain an acid solution with a concentration of more than 90 wt % and condensed water or a dilute acid solution; the acid solution with the concentration of more than 90 wt % obtained was recycled back to the step S1 as the acid solution; and the condensed water or the dilute acid solution obtained was recycled back to the step S3 as a secondary acidolysis solvent.

After two-stage acid hydrolysis, a glucose conversion rate of 72.76% and a xylose conversion rate of 61.74% could be achieved.

### Embodiment 7

A method for obtaining an acid-containing monosaccharide solution from a straw-type lignocellulosic biomass by acid treatment included the following steps.

In S1, wheat straw (40-100 mesh) was mixed with an aqueous sulfuric acid solution at a mass concentration of 60 wt %, and the wheat straw could be added in batches or in one portion, wherein a mass ratio of the wheat straw to the aqueous sulfuric acid solution was 1:1. The reaction liquid was subjected to first acidolysis reaction at room temperature and atmospheric pressure until the reaction liquid had good fluidity, so as to obtain a first acidolysis liquid.

In S2, the first acidolysis liquid obtained in the step S1 was fully mixed with tris(2-ethylhexyl)amine, wherein a mass ratio of the first acidolysis liquid to the tris(2-ethylhexyl)amine was 1:3. After uniform mixing, solid-liquid separation was performed to obtain an acid-containing liquid phase and a glycan-containing and lignin-containing residue.

In S3, water and/or an acid solution were added to the glycan-containing and lignin-containing residue obtained in the step S2 for mixing to obtain a secondary acidolysis mixture (with an acid concentration of 30 wt %), and then, secondary acidolysis reaction was performed at 100° C. and atmospheric pressure for 1.5 hour. After the acid reaction, solid-liquid separation was performed to obtain an acid-containing monosaccharide solution and a lignin precipitate. The acid-containing monosaccharide solution obtained was treated by a simulated moving bed to obtain a monosaccharide solution and an acid solution.

In S4, water was added to the acid-containing liquid phase obtained in the step S2 for back-extraction, and the mixture was allowed to stand for phase separation to obtain an acid solution and an organic phase. The organic phase obtained was purified and then recycled back to the step S2 as the organic solvent. The acid solution obtained (including the acid solution obtained in the step S3) was concentrated to obtain an acid solution with a concentration of more than 60 wt % and condensed water or a dilute acid solution; the acid solution with the concentration of more than 60 wt % obtained was recycled back to the step S1 as the acid solution; and the condensed water or the dilute acid solution obtained was recycled back to the step S3 as a secondary acidolysis solvent.

After two-stage acid hydrolysis, a glucose conversion rate of 84.53% and a xylose conversion rate of 76.85% could be achieved.

### Embodiment 8

A method for obtaining an acid-containing monosaccharide solution from a straw-type lignocellulosic biomass by acid treatment included the following steps.

In S1, wheat straw (40-100 mesh) was mixed with an aqueous sulfuric acid solution at a mass concentration of 90 wt %, and the wheat straw could be added in batches or in one portion, wherein a mass ratio of the wheat straw to the aqueous sulfuric acid solution was 1:2. The reaction liquid was subjected to first acidolysis reaction at room temperature and atmospheric pressure until the reaction liquid had good fluidity, so as to obtain a first acidolysis liquid.

In S2, the first acidolysis liquid obtained in the step S1 was fully mixed with tris(2-ethylhexyl)amine, wherein a mass ratio of the first acidolysis liquid to the tris(2-ethylhexyl)amine was 1:3. After uniform mixing, solid-liquid separation was performed to obtain an acid-containing liquid phase and a glycan-containing and lignin-containing residue.

In S3, water and/or an acid solution were added to the glycan-containing and lignin-containing residue obtained in the step S2 for mixing to obtain a secondary acidolysis mixture (with an acid concentration of 10 wt %), and then, secondary acidolysis reaction was performed at 80° C. and atmospheric pressure for 0.5 hour. After the acid reaction, solid-liquid separation was performed to obtain an acid-containing monosaccharide solution and a lignin precipitate. The acid-containing monosaccharide solution obtained was treated by a simulated moving bed to obtain a monosaccharide solution and an acid solution.

In S4, water was added to the acid-containing liquid phase obtained in the step S2 for back-extraction, and the mixture was allowed to stand for phase separation to obtain an acid solution and an organic phase. The organic phase obtained was purified and then recycled back to the step S2 as the organic solvent. The acid solution obtained (including the acid solution obtained in the step S3) was concentrated to obtain an acid solution with a concentration of more than 90 wt % and condensed water or a dilute acid solution; the acid solution with the concentration of more than 90 wt % obtained was recycled back to the step S1 as the acid solution; and the condensed water or the dilute acid solution obtained was recycled back to the step S3 as a secondary acidolysis solvent.

After two-stage acid hydrolysis, a glucose conversion rate of 88.21% and a xylose conversion rate of 82.46% could be achieved.

### Embodiment 9

A method for obtaining an acid-containing monosaccharide solution from a straw-type lignocellulosic biomass by acid treatment included the following steps.

In S1, wheat straw (40-100 mesh) was mixed with an aqueous sulfuric acid solution at a mass concentration of 80 wt %, and the wheat straw could be added in batches or in one portion, wherein a mass ratio of the wheat straw to the aqueous sulfuric acid solution was 1:1.5. The reaction liquid was subjected to first acidolysis reaction at room temperature and atmospheric pressure until the reaction liquid had good fluidity, so as to obtain a first acidolysis liquid.

In S2, the first acidolysis liquid obtained in the step S1 was fully mixed with 1-heptanol, wherein a mass ratio of the first acidolysis liquid to the 1-heptanol was 1:3. After uniform mixing, solid-liquid separation was performed to obtain an acid-containing liquid phase and a glycan-containing and lignin-containing residue.

In S3, water and/or an acid solution were added to the glycan-containing and lignin-containing residue obtained in the step S2 for mixing to obtain a secondary acidolysis mixture (with an acid concentration of 20 wt %), and then, secondary acidolysis reaction was performed at 90° C. and atmospheric pressure for 1 hour. After the acid reaction, solid-liquid separation was performed to obtain an acid-containing monosaccharide solution and a lignin precipitate. The acid-containing monosaccharide solution obtained was treated by a simulated moving bed to obtain a monosaccharide solution and an acid solution.

In S4, water was added to the acid-containing liquid phase obtained in the step S2 for back-extraction, and the mixture was allowed to stand for phase separation to obtain an acid solution and an organic phase. The organic phase obtained was purified and then recycled back to the step S2 as the organic solvent. The acid solution obtained (including the acid solution obtained in the step S3) was concentrated to obtain an acid solution with a concentration of more than 80 wt % and condensed water or a dilute acid solution; the acid solution with the concentration of more than 80 wt % obtained was recycled back to the step S1 as the acid solution; and the condensed water or the dilute acid solution obtained was recycled back to the step S3 as a secondary acidolysis solvent.

After two-stage acid hydrolysis, a glucose conversion rate of 85.64% and a xylose conversion rate of 78.49% could be achieved.

### Embodiment 10

A method for obtaining an acid-containing monosaccharide solution from a straw-type lignocellulosic biomass by acid treatment included the following steps.

In S1, wheat straw (40-100 mesh) was mixed with an aqueous sulfuric acid solution at a mass concentration of 80 wt %, and the wheat straw could be added in batches or in one portion, wherein a mass ratio of the wheat straw to the aqueous sulfuric acid solution was 1:1.5. The reaction liquid was subjected to first acidolysis reaction at room temperature and atmospheric pressure until the reaction liquid had good fluidity, so as to obtain a first acidolysis liquid.

In S2, the first acidolysis liquid obtained in the step S1 was fully mixed with methyl ethyl ketone, wherein a mass ratio of the first acidolysis liquid to the methyl ethyl ketone was 1:3. After uniform mixing, solid-liquid separation was performed to obtain an acid-containing liquid phase and a glycan-containing and lignin-containing residue.

In S3, water and/or an acid solution were added to the glycan-containing and lignin-containing residue obtained in the step S2 for mixing to obtain a secondary acidolysis mixture (with an acid concentration of 20 wt %), and then, secondary acidolysis reaction was performed at 90° C. and atmospheric pressure for 1 hour. After the acid reaction, solid-liquid separation was performed to obtain an acid-containing monosaccharide solution and a lignin precipitate. The acid-containing monosaccharide solution obtained was treated by a simulated moving bed to obtain a monosaccharide solution and an acid solution.

In S4, water was added to the acid-containing liquid phase obtained in the step S2 for back-extraction, and the mixture was allowed to stand for phase separation to obtain an acid solution and an organic phase. The organic phase obtained was purified and then recycled back to the step S2 as the organic solvent. The acid solution obtained (including the acid solution obtained in the step S3) was concentrated to obtain an acid solution with a concentration of more than 80 wt % and condensed water or a dilute acid solution; the acid solution with the concentration of more than 80 wt % obtained was recycled back to the step S1 as the acid solution; and the condensed water or the dilute acid solution obtained was recycled back to the step S3 as a secondary acidolysis solvent.

After two-stage acid hydrolysis, a glucose conversion rate of 83.42% and a xylose conversion rate of 75.93% could be achieved.

### Embodiment 11

A method for obtaining an acid-containing monosaccharide solution from a straw-type lignocellulosic biomass by acid treatment included the following steps.

In S1, wheat straw (40-100 mesh) was mixed with an aqueous sulfuric acid solution at a mass concentration of 80 wt %, and the wheat straw could be added in batches or in one portion, wherein a mass ratio of the wheat straw to the aqueous sulfuric acid solution was 1:1.5. The reaction liquid was subjected to first acidolysis reaction at room temperature and atmospheric pressure until the reaction liquid had good fluidity, so as to obtain a first acidolysis liquid.

In S2, the first acidolysis liquid obtained in the step S1 was fully mixed with tris(2-ethylhexyl)amine, wherein a mass ratio of the first acidolysis liquid to the tris(2-ethylhexyl)amine was 1:3. After uniform mixing, solid-liquid separation was performed to obtain an acid-containing liquid phase and a glycan-containing and lignin-containing residue.

In S3, water and/or an acid solution were added to the glycan-containing and lignin-containing residue obtained in the step S2 for mixing to obtain a secondary acidolysis mixture (with an acid concentration of 20 wt %), and then, secondary acidolysis reaction was performed at 90° C. and atmospheric pressure for 1 hour. After the acid reaction, solid-liquid separation was performed to obtain an acid-containing monosaccharide solution and a lignin precipitate. The acid-containing monosaccharide solution obtained was treated by a simulated moving bed to obtain a monosaccharide solution and an acid solution.

In S4, water was added to the acid-containing liquid phase obtained in the step S2 for back-extraction, and the mixture was allowed to stand for phase separation to obtain an acid solution and an organic phase. The organic phase obtained was purified and then recycled back to the step S2 as the organic solvent. The acid solution obtained (including the acid solution obtained in the step S3) was concentrated to obtain an acid solution with a concentration of more than 80 wt % and condensed water or a dilute acid solution; the acid solution with the concentration of more than 80 wt % obtained was recycled back to the step S1 as the acid solution; and the condensed water or the dilute acid solution obtained was recycled back to the step S3 as a secondary acidolysis solvent.

After two-stage acid hydrolysis, a glucose conversion rate of 91.58% and a xylose conversion rate of 85.29% could be achieved. Meanwhile, the total sugar concentration of the final monosaccharide solution could reach up to 109.61 g/L.

### Embodiment 12

A method for obtaining an acid-containing monosaccharide solution from a straw-type lignocellulosic biomass by acid treatment was the same as that in Embodiment 1, except that a different solvent was used for the treatment.

| | Cellulose | Hemicellulose |
|---|---|---|
| Organic | conversion | conversion |
| solvent | rate | rate |
| Methanol | 52.34% | 38.13% |
| Ethanol | 54.29% | 39.46% |
| Acetone | 55.31% | 41.22% |
| Polyacetone | 49.57% | 33.19% |
| Triethylamine | 69.85% | 57.61% |
| Isopropylamine | 63.49% | 50.86% |

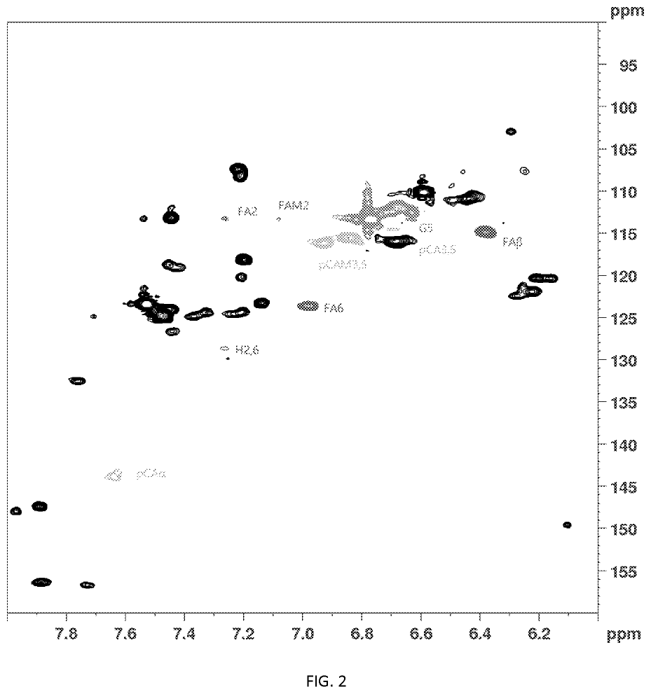

FIG. 2 shows an aromatic structure of a residual lignin component after secondary acidolysis, which may be recycled for preparing a bio-based fertilizer.

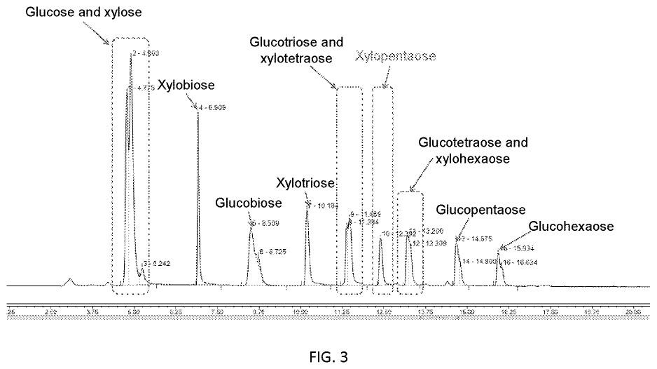

FIG. 3 shows types and distribution of glycans obtained by primary acidolysis, which are mainly glucan and xylan.

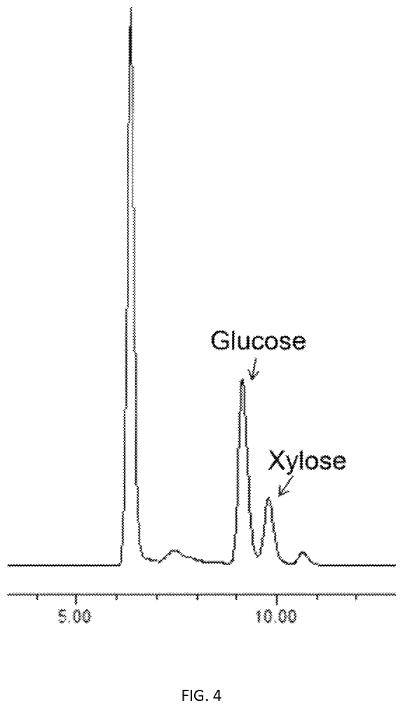

FIG. 4 shows a monosaccharide solution obtained by the present invention, wherein components of the solution are mainly glucose and xylose.

The present invention provides an idea and a method for a method for obtaining an acid-containing monosaccharide solution from a straw-type lignocellulosic biomass by acid treatment, with many methods and ways to realize the technical solution specifically. Those described above are merely the preferred embodiments of the present invention, and it should be pointed out that those of ordinary skills in the art may further make improvements and decorations without departing from the principle of the present invention, and these improvements and decorations should also be regarded as the scope of protection of the present invention. All the unspecified components in the embodiments can be realized by the prior art.

What is claimed is:

1. A method for preparing a monosaccharide from a lignocellulosic biomass, comprising the following steps:
S1: subjecting the lignocellulosic biomass to a first acidolysis reaction catalyzed by an acid solution with a concentration of more than 50 wt % at 20-30° C. to obtain a first acidolysis liquid;
S2: evenly mixing the first acidolysis liquid with an organic solvent, and performing solid-liquid separation to obtain a glycan-containing residue; and
S3: adding water and/or an acid solution to the glycan-containing residue to obtain a mixture with an acid concentration of less than 45 wt %, and performing a second acidolysis reaction to obtain a monosaccharide-containing reaction liquid;
wherein in the step S2, the organic solvent is anyone of tri(2-ethylhexyl)amine, triethylamine, and isopropylamine.

2. The method according to claim 1, wherein, in the step S1, the lignocellulosic biomass comprises a straw-type lignocellulosic biomass.

3. The method according to claim 2, wherein, the straw-type lignocellulosic biomass is any one or a combination of several of wheat straw, corn stover, rice straw, sorghum straw, bagasse, corn cob, switchgrass, reed and rapeseed.

4. The method according to claim 1, wherein, in the step S1, the aqueous acid solution is an aqueous solution of an inorganic acid; and the acid concentration in the acid solution is 55 wt %-95 wt %.

5. The method according to claim 1, wherein, the inorganic acid is any one or a combination of several of sulfuric acid, hydrochloric acid and phosphoric acid.

6. The method according to claim 5, wherein, in the step S1, the acid concentration in the acid solution is 60 wt %-90 wt %.

7. The method according to claim 1, wherein, in the step S1, a mass ratio of the lignocellulosic biomass to an acid in the acid solution is 1:0.5-2.5.

8. The method according to claim 1, wherein, a reaction temperature of the first acidolysis reaction is room temperature. a pressure of the first acidolysis reaction is atmospheric pressure.

9. The method according to claim 1, wherein, in the step S2, a mass ratio of the first acidolysis liquid to the organic solvent is 1: 1-5.

10. The method according to claim 1, wherein, in the step S3, the acid concentration of the mixture is 5 wt %-35 wt %.

11. The method according to claim 1, wherein, in the step S3, the acid concentration of the mixture is 10 wt %-30 wt %.

12. The method according to claim 1, wherein, in the step S3, a temperature of the second acidolysis reaction is 60-120° C.

13. The method according to claim 1, wherein, in the step S3, a temperature of the second acidolysis reaction is 80-100° C.

14. The method according to claim 1, wherein, in the step S3 a pressure of the second acidolysis reaction is atmospheric pressure.

15. The method according to claim 1, wherein, in the step S3, the second acidolysis reaction lasts for 0.3-2.5 hours.

16. The method according to claim 1, wherein, in the step S3, the monosaccharide-containing reaction liquid is subjected to the solid-liquid separation to obtain a monosaccharide-containing solution and a lignin precipitate; and the monosaccharide-containing solution is treated by a simulated moving bed to obtain a monosaccharide liquid and an acid solution.

17. The method according to claim 1, wherein the method further comprises the following step:
S4: adding water to an acid-containing liquid phase obtained after the solid-liquid separation in the step S2 for back-extraction, and allowing the mixture to stand for phase separation to obtain an acid solution and an organic phase.

18. The method according to claim 17, wherein the organic phase obtained in the step S4 is purified and then recycled back to the step S2 as the organic solvent.

19. The method according to claim 17, wherein the acid solution obtained in the step S4 is concentrated to obtain an acid solution with a concentration of more than 50 wt % and condensed water or a dilute acid solution.

20. The method according to claim 19, wherein the acid solution with the concentration of more than 50 wt % obtained is recycled back to the step S1 as the acid solution; and the condensed water or the dilute acid solution obtained is recycled back to the step S3.
